(19) United States
(12) Patent Application Publication
(10) Pub. No.: US 2025/0218565 A1
(43) Pub. Date: Jul. 3, 2025

(54) LIFESTYLE IMPROVEMENT ASSISTANCE SYSTEM AND LIFESTYLE IMPROVEMENT ASSISTANCE PROGRAM

(71) Applicant: NATIONAL UNIVERSITY CORPORATION TOKAI NATIONAL HIGHER EDUCATION AND RESEARCH SYSTEM, Aichi (JP)

(72) Inventor: Sumio YAMADA, Aichi (JP)

(73) Assignee: NATIONAL UNIVERSITY CORPORATION TOKAI NATIONAL HIGHER EDUCATION AND RESEARCH SYSTEM, Aichi (JP)

(21) Appl. No.: 18/849,988

(22) PCT Filed: Mar. 23, 2023
(86) PCT No.: PCT/JP2023/011372

§ 371 Date: Sep. 23, 2024

(30) Foreign Application Priority Data
Mar. 25, 2022 (JP) 2022-050382

(51) Int. Cl. G16H 20/60; A61B 5/145; G16H 10/60; G16H 20/30

(52) U.S. Cl. CPC G16H 20/60; A61B 5/14532; G16H 10/60; G16H 20/30

(57) ABSTRACT

The present disclosure provides a technology capable of appropriately supporting improvement of an individual's lifestyle. The lifestyle improvement support system is provided with an acquisition device capable of acquiring from a target person at least their blood glucose level data, physical activity data, and dietary activity data, as well as a controlling device.

### TECHNICAL FIELD

The present disclosure relates to a lifestyle improvement support system and a lifestyle improvement support program. This application claims priority to Japanese Patent Application No. 2022-050382, filed Mar. 25, 2022, and the entire content of the priority Japanese application are incorporated herein by reference.

### BACKGROUND ART

With the rapid progress of aging, the proportion of lifestyle-related diseases among all diseases is increasing. Lifestyle-related diseases are roughly classified into cardiovascular diseases, such as myocardial infarction and cerebral infarction, and diseases that are risk factors for cardiovascular diseases, such as diabetes, hypertension, and dyslipidemia. Because there is a continuum between the risk factors and the onset of cardiovascular disease, it is said to be the biggest obstacle to healthy life expectancy. After any needed medical or surgical treatment, it is important for patients who have developed cardiovascular disease to administer their medication or improve their lifestyles, including exercise and diet, from the perspective of preventing a relapse and reintegrating into society. Patients improving their lifestyles is important not only for the patients themselves, as described above, but also from the viewpoint of preventing lifestyle-related disease. In recent years, the development of appropriate teaching technology (or supporting technology) for improving a person's lifestyle has been actively carried out accompanying the related circumstances. For example, the following Patent Document 1 disclosed a technology related to dietary guidance for improving the target person's eating habits.

### PRIOR ART DOCUMENTS

### Patent Documents

Patent Document 1: Japanese Patent Application Publication No. 2007-133525

### SUMMARY OF THE INVENTION

The lifestyle type that needs to be improved often varies from individual to individual. Therefore, in the guidance (or support) for lifestyle improvement, it is important to propose items for lifestyle improvement that are suitable for each individual. The present disclosure is made in consideration of the related circumstances, and the main objective thereof is to provide technology capable of appropriately supporting each individual improving their lifestyle.

To achieve the relevant objective, the present disclosure provides a lifestyle improvement support system that supports improving a target person's blood glucose level by improving the target person's lifestyle. The above-specified lifestyle improvement support system is provided with a data acquisition device capable of acquiring at least blood glucose level data, physical activity data, and dietary activity data, as well as a controlling device. The above-specified controlling device is characterized by being configured to perform:
extracting a plurality of factors, including a factor related to the target person's physical activity and a factor related to the target person's dietary activity, that the target person should improve, based on at least the above-specified physical activity data and dietary activity data;
selecting one or more relevant factors from among the plurality of the above-specified extracted factors;
notifying the target person about the above-specified selected one or more factors to stimulate the target person to improve their lifestyle in relation to each factor;
determining whether each factor improved by the target person contributed to improving the target person's blood glucose level based on at least the blood glucose level data acquired from the target person before the improvement of the target person's lifestyle and the blood glucose level data acquired from the target person after stimulating the target person to improve their lifestyle; and
notifying the target person about one or more factors from among the above-specified factors that were determined to have contributed to improving the target person's blood glucose level in order to stimulate the target person to improve their lifestyle in relation to each factor. The lifestyle improvement support system is configured to notify the target person about the factors that should be improved by the target person and the like based on whether the target person's blood glucose level data improved (in other words, based on the target person's specific data). Therefore, the lifestyle improvement support system can appropriately support improving a target person's lifestyle.

In a preferred embodiment of the lifestyle improvement support system disclosed in this specification, the above-described acquisition device is configured to be able to acquire additional health data, and the above-specified controlling device is configured to perform extracting a plurality of factors, including factors related to the target person's physical activity and the factors related to the target person's dietary activity, that the above-specified target person should improve based on at least the above-specified physical activity data, the above-specified dietary activity data, and the above-specified health data. Since more suitable factors for the target person can be extracted by considering the target person's health data when extracting the factors, considering the target person's health data is preferable.

In the preferred embodiment of the lifestyle improvement support system disclosed in this specification, the above-described acquisition device is configured to be able to further acquire self-efficacy, and the above-described controlling device is configured to notify the above-specified target person about the plurality of the above-specified extracted factors, to acquire the target person's self-efficacy to improve their lifestyle in relation to one or more of the notified factors, and to select one or more of the above-specified factors after taking the self-efficacy into account. Since the more suitable factors for the target person can be extracted by considering the target person's self-efficacy when extracting the factors, considering the target person's self-efficacy is preferable.

From another perspective, in the above-specified controlling device of the lifestyle improvement support system subject to the present disclosure, a lifestyle improvement support program is provided, wherein the program executes:
extracting a plurality of factors, including a factor related to the target person's physical activity and a factor related to the target person's dietary activity, that the above-specified target person should improve, based on at least the above-specified physical activity data and the above-specified dietary activity data;
selecting one or more relevant factors from among the above-described extracted factors;
notifying the above-specified target person about the above-specified selected one or more factors to stimulate the target person to improve their lifestyle in relation to each factor;
determining whether each factor improved by the above-specified target person contributed to improving the target person's blood glucose level based on at least the above blood glucose level data acquired from the above-specified target person before the improvement of the lifestyle described above and the blood glucose level data acquired from the above-specified target person after stimulating the above-specified target person to improve their lifestyle; and
notifying the target person about one or more factors from among the above-specified factors that were determined to have contributed to improving the target person's above-specified blood glucose level to stimulate the above-specified target person to improve their lifestyle in relation to each factor.

### BRIEF DESCRIPTION OF DRAWINGS

FIG. 1 is a conceptual diagram illustrating a lifestyle improvement support system according to one embodiment.

FIG. 2 is a block diagram illustrating the lifestyle improvement support system according to the one embodiment.

FIG. 3 is a diagram explaining a first lifestyle improvement support method using the lifestyle improvement support system according to the one embodiment.

FIG. 4 is a flowchart illustrating a procedure for the first lifestyle improvement support method using the lifestyle improvement support system according to the one embodiment.

FIG. 5 is a flowchart illustrating a procedure whereby “excessive intake of calories” is extracted as a factor according to the one embodiment.

FIG. 6 is a flowchart illustrating a procedure whereby “unbalanced nutrition” is extracted as a factor according to the one embodiment.

FIG. 7 is a flowchart illustrating a procedure whereby “late-night meal” is extracted as a factor according to the one embodiment.

FIG. 8 is a flowchart illustrating a procedure whereby the “time gap with a previous meal” is extracted as a factor according to the one embodiment.

FIG. 9 is a flowchart illustrating a procedure whereby “excessive performance of SB during 30 minutes after a meal” is extracted as a factor according to the one embodiment.

FIG. 10 is a flowchart illustrating a procedure whereby “excessive performance of Sedentary Behavior's (SB) in a day” is extracted as a factor according to the one embodiment.

FIG. 11 is a flowchart illustrating a procedure whereby “lacking performance of Moderate-to-Vigorous Physical Activity (MVPA) in a day” is extracted as a factor according to the one embodiment.

FIG. 12 is a flowchart illustrating a procedure that determines the target person's blood glucose level is improved according to the one embodiment.

FIG. 13 is a diagram explaining a second lifestyle improvement support method using the lifestyle improvement support system according to one embodiment.

FIG. 14 is a flowchart illustrating a procedure for the second lifestyle improvement support method using the lifestyle improvement support system according to the one embodiment.

### DESCRIPTION OF THE EMBODIMENTS

Preferred embodiments of the lifestyle improvement support system and the lifestyle improvement support program disclosed in this specification are described below with reference to the drawings as needed. Matters other than those specifically mentioned in the specification and necessary for the implementation of the present disclosure can be understood as matters of design for a person skilled in the art based on the prior art in the field. The present disclosure may be implemented in accordance with the teachings disclosed in the present specification and common general knowledge in the art. It should be noted that the following description is not intended to limit the technology disclosed in this specification to the following embodiments. In the present specification, the expression “A to B” indicating a numerical range means A or more and B or less. Accordingly, the situation where the value is greater than A and less than B is included.

Next, in the present specification and claims, a lifestyle improvement related to a factor can mean a behavior's performance that is expected to improve the factor when performed by a majority of target persons. For example, when “late-night meals” is cited as a factor, “eating dinner before a predetermined time” is cited as a lifestyle improvement related to the factor.

### <Lifestyle Improvement Support System>

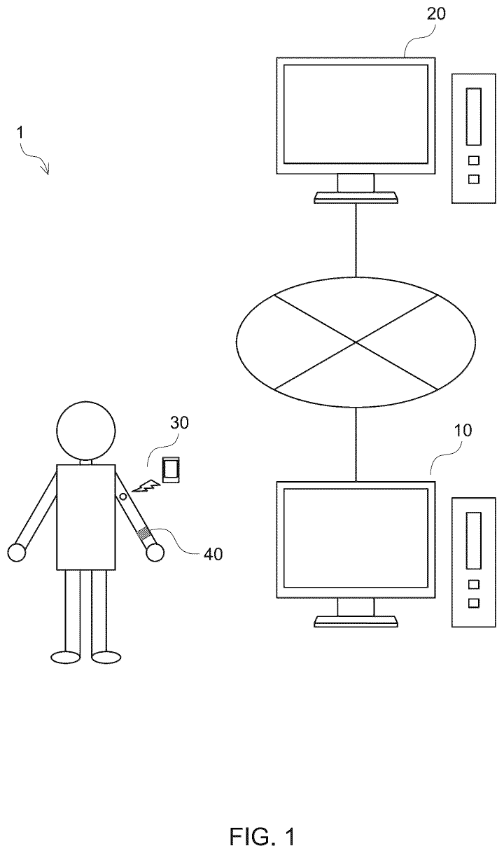

First, the lifestyle improvement support system 1 according to the present embodiment is described. FIG. 1 is a conceptual diagram illustrating the lifestyle improvement support system according to the present embodiment. The lifestyle improvement support system 1 according to the present embodiment is a system for supporting the improvement of a target person's lifestyle (specifically, improvement of the target person's blood glucose level by improving their lifestyle). As shown in FIG. 1, the lifestyle improvement support system 1 includes a target person's terminal 10 and a management server 20. The target person's terminal 10 and the management server 20 are described in detail below.

### (Target Person's Terminal 10)

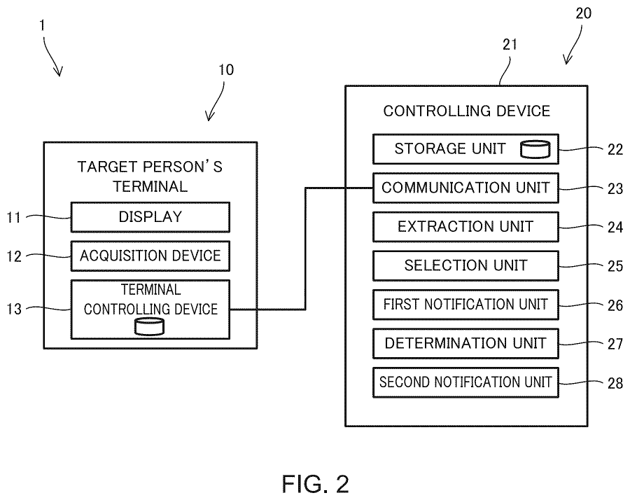

The target person's terminal 10 is a terminal used by the target person. The target person's terminal 10 may be, for example, a laptop personal computer, or a smartphone or tablet terminal, used by the target person. As shown in FIG. 2, the target person's terminal 10 includes a display 11, an acquisition device 12, and a terminal controlling device 13. For example, the acquisition device 12 includes an input unit that acquires data input by the target person using a keyboard, mouse, or touch panel, or the like, and an external device communication unit. The external device communication unit acquires data by wireless communication (such as Bluetooth (registered trademark) or Wi-Fi (registered trademark)) from a continuous blood glucose meter 30 and a sensor device 40, described later. The terminal controlling device 13 is connected to the display 11 and the acquisition device 12 enabling communication between the terminal controlling device 13 and the display 11 or the acquisition device 12.

The acquisition device 12 according to the present embodiment is configured to be able to acquire the target person's blood glucose level data, physical activity data, dietary activity data, health data, and self-efficacy. All the data is described below.

The blood glucose data is data related to a target person's blood glucose level (typically, a blood glucose level in blood). The blood glucose data can be used as an indicator to assess whether there is an improvement in blood glucose level. In the present disclosure, the blood glucose level is closely related to diseases such as diabetes, cerebral infarction, and heart disease. Therefore, the lifestyle improvement based on blood glucose data may be highly effective from the perspective of maintaining or improving the target person's health. Since the blood glucose level data is specific to the target person, it is considered that the blood glucose level data can propose a suitable lifestyle improvement for each target person. Examples of such blood glucose level data include TIR (Time In Range), Mean Amplitude of Glycemic Excursions (MAGE), Postprandial hyperglycemia (PHG), Mean Self-monitoring Glucose level (MSG), and the like.

The TIR indicates the percentage of the day the blood glucose level is within the target range (typically between 70 and 180 mg/dl). It is said that a higher TIR is preferable, and for example, by comparing the TIR measurements, it is possible to evaluate whether the blood glucose level is improved. In this specification, it is preferable for the TIR to be 60% or more, more preferable for the TIR to be 70% or more, and even more preferable for the TIR to be 80% or more (for example, more than 80%). When the TIR is more than 80%, it can be associated with HbA1c (hemoglobin A1c) being less than 6.5% (the target for glycemic control). The Mean Amplitude of Glycemic Excursions indicates an average value (mg/dl) of glycemic excursions that is obtained only by a glycemic excursion equal to or greater than the standard deviation in the target person's blood glucose level in one day. The Mean Amplitude of Glycemic Excursions is obtained by filtering out small excursions whose amplitude does not exceed a certain threshold (e.g., 1 SD), and reflects the excursions in the blood glucose independent of overall levels. It is preferable for the Mean Amplitude of Glycemic Excursions to be lower, and it can be used to evaluate whether the blood glucose level is improved by comparing the Mean Amplitude of Glycemic Excursions measurements. The postprandial hyperglycemia indicates a state when the blood glucose level during the 2 hours after a meal is higher than, for example, 200 mg/dl. It is preferable that the state of postprandial hyperglycemia is controlled, and for example, by comparing the presence or absence of such suppression, it is possible to evaluate the presence or absence of improvement in blood glucose level. The average blood glucose level represents the average value of the blood glucose level for one day. It is preferable the average blood glucose level is within a predetermined range (typically, about 100 mg/dl to 120 mg/dl), and for example, by comparing the difference between the average blood glucose level and the target level, it is possible to evaluate whether the target person's blood glucose level has improved.

As shown in FIG. 1, in the present embodiment, a sensor attached to a continuous blood glucose meter (continuous blood glucose monitor) 30 is attached to the target person's upper arm, and the blood glucose level data is acquired by scanning the sensor with the continuous blood glucose monitor at a predetermined time. The above-specified commercially continuous blood glucose meter can be used without any particular limitation. The blood glucose level data can be obtained by, for example, periodically collecting blood. In the present embodiment, the blood glucose level data is transmitted from the continuous blood glucose meter 30 to the external device communication unit by wireless communication.

The physical activity data is data related to the target person's activity mode. Examples of such physical activity data include data on: pulse rate (times/minute); activity intensity (METs); time (minutes/day) during which physical activity (such as, SB and MVPA) is performed; sleep time; and the like, which are acquired by a sensor device (typically, a sensor device having functions including a triaxial acceleration sensor and a pulse sensor). METs is a unit of exercise intensity, and indicates the intensity of an activity calculated by the number of times the energy is consumed compared to when the resting state is set to 1. The target person's SB can also mean low intensity physical activity on the order of 1.5 METs or less (including decubitus, sitting and standing). MVPA can mean moderate to high intensity physical activity that is light breath-taking activity (in the order of about 3.0 METs) or more (such as, brisk walking). From the viewpoint of keeping blood glucose levels within an appropriate range, it is preferable to perform SB for a shorter time, and to perform MVPA for a longer time.

As shown in FIG. 1, in the present embodiment, the physical activity data is acquired by attaching the sensor device 40 to the target person's wrist. As such, a commercially available sensor device can be used without any particular limitation. The sensor device may be attached to the target person's ankle or the like. In the present embodiment, the physical activity data is transmitted from the sensor device 40 to the external device communication unit by wireless communication.

The dietary activity data is data related to the target person's dietary mode. Examples of such dietary activity data include whether each meal (breakfast, lunch, or dinner) was consumed, the intake time of each meal, the food material consumed in each meal, the amount of intake (kcal), the ratio of nutrients (such as, protein, fat, carbohydrate, and the like), and the data on dietary behavior. In this specification, the dietary behavior is an item indicating the target person's eating speed and whether the target person bites and eats the dietary staples and accompanying dishes separately. In the present embodiment, the above-described items are displayed on the display 11, and the target person inputs an answer to the items through a keyboard.

The health data is data related to the target person's personal information. Examples of such health data mean the target person's personal data, such as the target person's sex, age, height, weight, medical history, and whether the target person taking a prescription drug, and if so, the type of the prescription drugs the target person is taking, and the like. Whether the target person is taking a prescription drug and the type of the prescription drug the target person is taking mean, for example, whether a target person took a hypoglycemic drug on the day or the previous day. Since the more suitable factors for the target person can be extracted by considering the target person's health data when extracting the factors, considering the health data is preferable. Specifically, for example, when the target person has a medical history of low back pain, knee pain, or the like, the target person performing excessive physical activity is not preferable. Thus, the factor, “insufficient performance of MVPA in a day” is not extracted, and instead, the setting is such that “performing insufficient light physical activity” can be extracted. In the present embodiment, the above-described items are displayed on the display 11, and the target person inputs an answer to the items using a keyboard.

The self-efficacy (SE) means the target person's self-efficacy for the lifestyle improvements related to the factor. The self-efficacy, as used in the present specification and the claims, means the target person recognizing their ability to successfully perform the behaviors required in a given situation. In other words, the self-efficacy is the degree of efficacy expectation a target person has, and the degree of confidence a target person has that they can perform the appropriate behaviors to produce a certain outcome. Additionally, the self-efficacy is a subjective indicator that includes psychological components and does not guarantee that the required behavior can actually be performed. Since the more suitable factors for the target person can be extracted by considering the target person's self-efficacy when extracting the factors, considering the self-efficacy is preferable. That is, it is preferable to consider the target person's self-efficacy because the lifestyle improvement support method described later can be performed without delay. For example, the index of the self-efficacy may have four levels: “1. Strongly believe it is feasible,” “2. Believe it is feasible,” “3. Do not know whether it is feasible,” and “4. Believe it is not feasible.” Further, for example, when the above-mentioned 1 or 2 level is input, it can be determined the target person's self-efficacy is high. Additionally, it is not intended to be limited thereto. In the present embodiment, the evaluation items for evaluating the target person's self-efficacy for the lifestyle improvements related to each factor in the above-described four levels are displayed on the display 11, and the target person inputs an answer to the items using a keyboard.

### (Management Server 20)

The management server 20 is connected to the target person's terminal 10 enabling communication. The management server 20 may be configured by a single computer or a plurality of computers cooperating with each other. The management server 20 has a controlling device 21. Although not shown in FIG. 2, in the present embodiment, the management server 20 has a display unit and an input unit, similar to the target person's terminal 10.

The configuration of the controlling device 21 is not particularly limited. For example, the controlling device 21 is a microcomputer. For example, the controlling device 21 has an I/F (Interface), a CPU (Central Processing Unit), a ROM (Read Only Memory), and a RAM (Random Access Memory). As shown in FIG. 2, in the present embodiment, the controlling device 21 has a storage unit 22 and a communication unit 23. The controlling device 21 further has an extraction unit 24, a selection unit 25, a first notification unit 26, a determination unit 27, and a second notification unit 28. The units 22 to 28 included in the controlling device 21 may be configured by one or more processors or may be configured by a circuit.

The communication unit 23 is configured to be able to communicate with the target person's terminal 10 used by the target person. The communication unit 23 is connected to the terminal controlling device 13 of the target person's terminal 10 enabling communication. The communication unit 23 is configured to be able to transmit and receive various information to and from the target person's terminal 10. The other parts are described in the description of the lifestyle improvement support method described later.

Next, an example of the lifestyle improvement support method using the lifestyle improvement support system 1 according to the present embodiment is described. It should be noted that the following description is not intended to limit the lifestyle improvement support method disclosed in this specification to the following embodiments. The lifestyle improvement support method disclosed in this specification may further include other steps at any stage, or the order of the steps may be changed.

### <First Lifestyle Improvement Support Method>

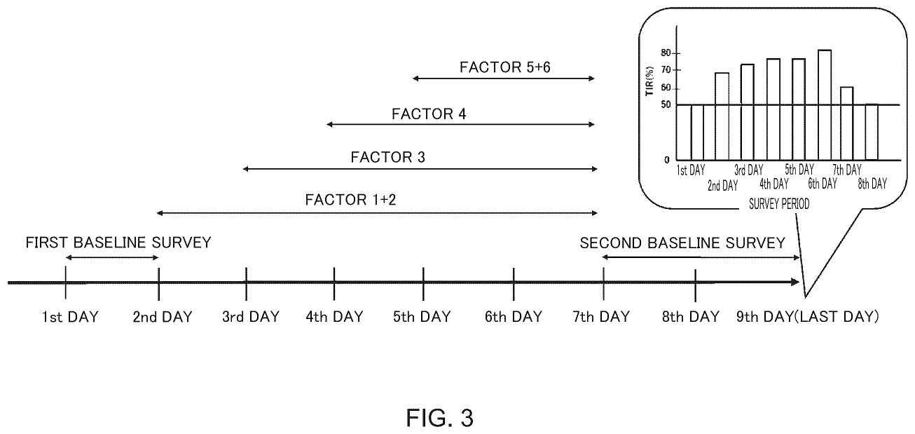

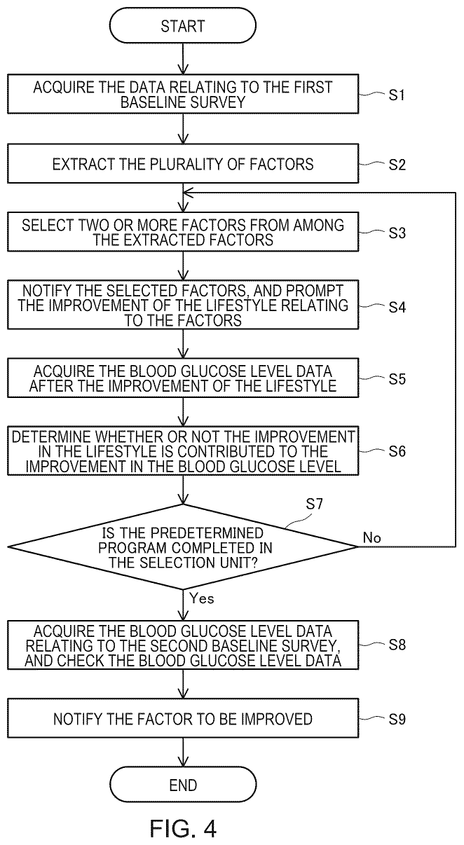

First, the first lifestyle improvement support method is described. FIG. 3 is a diagram explaining the first lifestyle improvement support method using a lifestyle improvement support system according to the present embodiment. FIG. 4 is a flowchart illustrating a procedure of the first lifestyle improvement support method using the lifestyle improvement support system according to the present embodiment. The first lifestyle improvement support method is described below with reference to the drawings as needed. Note the first lifestyle improvement support method is performed by a target person A, as one target person. Further, during the period from the first day to the ninth day in FIG. 3, the target person A has the sensor attached to the continuous blood glucose meter 30 on the upper arm and has the sensor device 40 on the wrist (see FIG. 1).

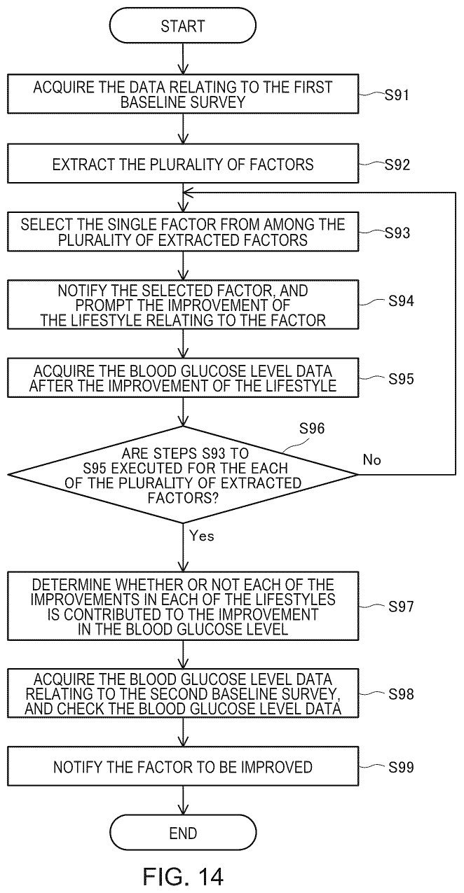

First, the first day in FIG. 3 is described with reference to FIG. 4. Initially, in Step S1 of FIG. 4, data related to a first baseline survey for the target person is acquired. The baseline survey may mean a survey on the target person's living conditions when the lifestyle improvement related to the factor is not implemented. In the first lifestyle improvement support method, the baseline surveys are conducted in Steps S1 and S8 of FIG. 4, and these are called the first baseline survey and a second baseline survey, respectively. Also, the same applies to Steps S91 and S98 in FIG. 14 described later.

For example, in the first baseline survey, the target person A's blood glucose level data, physical activity data, dietary activity data, and health data are obtained. Specifically, the acquisition device 12 of FIG. 2 respectively acquires the blood glucose level data and the physical activity data from the continuous blood glucose meter 30 and the sensor device 40 attached to the target person A. The acquisition device 12 also acquires the dietary activity data and the health data input by the target person A. It is preferable to also acquire the dietary activity data of the day before the first baseline survey, to use the dietary activity data when extracting factors described later. The terminal controlling device 13 transmits the various data acquired by the acquisition device 12 to the communication unit 23, and the various data are stored in the storage unit 22.

Next, in Step S2 of FIG. 4, a plurality of factors about the target person are extracted. Specifically, the extraction unit 24 of FIG. 2 extracts the plurality of factors related to the physical activity and the dietary activity to be improved by the target person A, based on the physical activity data, the dietary activity data, and the health data acquired in Step S1. In the present embodiment, the factors are extracted from among seven candidate factors (specifically, “excessive intake of calories,” “unbalanced nutrition,” “time gap with a previous meal,” “late night meal,” “excessive performance of SB during 30 minutes after a meal,” “excessive performance of SB in a day,” and “insufficient performance of MVPA in a day”) that are assumed by the present inventor to be easily involved in the fluctuation of blood glucose level.

In the present embodiment, a factor from the seven types of candidate factors described above is extracted, but the present invention is not limited thereto. The number of candidate factors is, for example, five types or more, and preferably seven types or more, from the viewpoint of extracting factors more suitable for the target person. On the other hand, the upper limit of the number of the candidate factors is, for example, twenty types or less; preferably fifteen types or less, and even more preferably ten types or less, from the viewpoint of performing factor extraction in a shorter time. Additionally, the number of candidate factors is not limited thereto. The types of the candidate factors are not limited to what is described above, and can be designed by a person skilled in the art as appropriate. Examples of other types of candidate factors include “lack of dietary fiber,” “eating fast,” “skipping a previous meal,” and “the excessive performance of SB in the 30-60 minutes after eating.” An example of a flowchart indicating the seven types of the candidate factors are extracted as factors is described below.

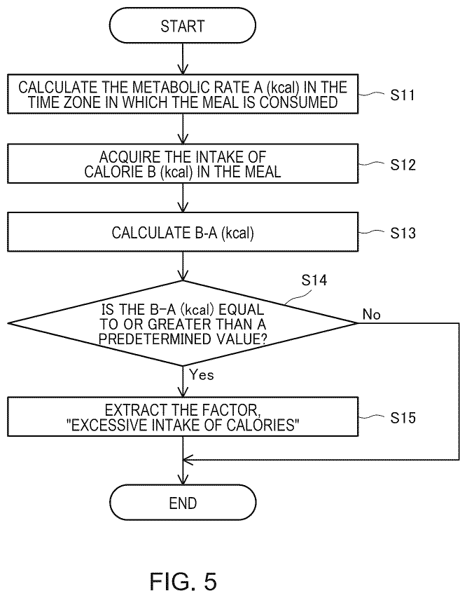

An example of a flowchart indicating “excessive intake of calories,” a candidate factor, is extracted as a factor is described. FIG. 5 is a flowchart illustrating a procedure whereby “the excessive intake of calories” is extracted as a factor. Although not shown in FIG. 5, the intake time when the target person had each meal (i.e., breakfast, lunch, or dinner) is first acquired. Next, based on the intake time of each of the target person's meals, the target person's time periods (from what time to what time) corresponding to: (1) the morning time range (the time period from the target person's wake-up time until their completion of the lunch intake); (2) the afternoon time range (the time period from the target person's completion of the lunch intake until their completion of the dinner intake); and (3) the night time range (the time period from the target person's completion of the dinner intake until their wake-up time), are calculated, and how many hours (hereinafter also referred to as P (time)) corresponds to the target person's each time period is calculated. Next, the target person's physical activity level Q in each time range is acquired. For example, the physical activity level when the target person performs physical activity at a low intensity is set to level 1.5, the physical activity when the target person performs physical activity at a medium intensity is set to level 1.75, and the physical activity when the target person performs physical activity at a high intensity is set to level 2.0. The flow is described when breakfast is used as an example of the meals. The present embodiment's lifestyle improvement support system is configured to also perform the same flow for lunch and dinner.

In step S11, (1) the target person's metabolic rate A (kcal) in the morning time range is calculated. For example, the metabolic rate A is calculated based on the following equation: (target person's basal metabolic rate)×(P/24)×Q. For example, the basal metabolic rate is calculated based on the following equation: the basal metabolic rate=[0.0481×body weight (kg)+0.0234×height (cm)−0.0138×age (year)−(constant)]×1,000÷4.186. The above constant can be defined as 0.4235 for men and 0.9708 for women. The method of calculating the metabolic rate A is not limited to this formula, and may be changed as appropriate.

In step S12, the target person's intake of calories B (kcal) at their breakfast is acquired. In Step S13, the target person's B-A (kcal) is calculated. In Step S14, it is determined whether the B-A (kcal) is equal to or greater than a predetermined value. It is preferable such value is appropriately set for each target person. For example, the value can be set to about 200 kcal, or it can also be set to about 100 kcal or about 50 kcal from the viewpoint of extracting “the excessive intake of calories” with a high accuracy. When the value of the B-A (kcal) is equal to or greater than the predetermined value (i.e., YES) in Step S14, the procedure proceeds to Step S15. In this case, the factor, “the excessive intake of calories” at breakfast, is extracted. Alternatively, when the value of B-A (kcal) is lower than the predetermined value (i.e., NO) in Step S4, the procedure proceeds to Step S15. The present embodiment's lifestyle improvement support system is configured to also perform the same flow for lunch and dinner.

In the above description, “the excessive intake of calories” at each of breakfast, lunch, and dinner is set as the factor candidate. Note, the present embodiment's lifestyle improvement support system is not limited to this factor candidate. For example, “the excessive intake of calories in a day” based on the intake of calories and the metabolic rate in a whole day can be set as the factor candidate.

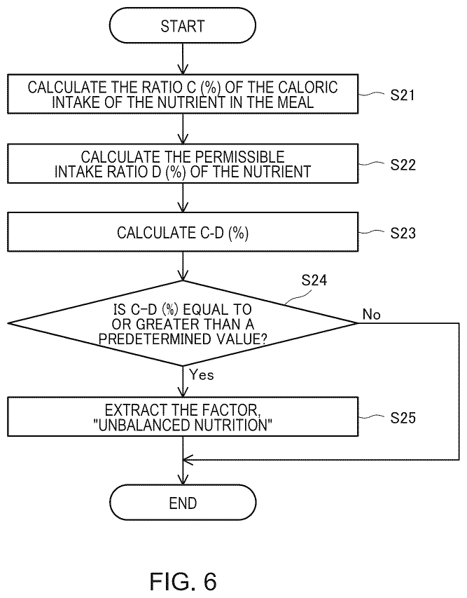

Next, an example of a flowchart indicating “the unbalanced nutrition” of the candidate factor, is extracted as a factor is described. FIG. 6 is a flowchart illustrating a procedure whereby “the unbalanced nutrition” is extracted as the factor. The flow is described when dinner is used as an example of the meals. The present embodiment's lifestyle improvement support system is configured to also perform the same flow for breakfast and lunch. As shown in FIG. 6, first, in Step S21, the ratio C (%) of the caloric intake of a nutrient by the target person's dinner is calculated. A carbohydrate is often extracted for a diabetic holder as such nutrient. Note, the present embodiment's lifestyle improvement support system is not limited to a carbohydrate, and other nutrients, such as proteins and lipids, might also be extracted. In the present embodiment, a case where the nutrient is a carbohydrate is described. In this case, in Step S21, (the caloric intake of the carbohydrate by dinner)/(caloric intake by dinner) (%) is calculated. Next, in Step S22, the permissible intake ratio D (%) for the carbohydrate by the dinner is calculated. For example, the permissible intake ratio D (%) is calculated as (the target person's metabolic rate during the night time range)×X/(the caloric intake by the dinner) (%). In this case, it is preferable to change X as appropriate depending on the nutrient's type. For example, in the case where the nutrient is a carbohydrate as in the present embodiment, X can be set to 0.45 to 0.55. On the other hand, for example, when the nutrient is a protein, X can be set to 0.2 to 0.3, or another example, when the nutrient is a lipid, X can be set to 0.15 to 0.25. In this manner, the permissible intake ratio D (%) of the carbohydrate based on metabolic rate in the night time range can be calculated.

Next, in Step S23, C-D (%) is calculated. Further, in Step S24, it is determined whether C-D (%) is equal to or greater than a predetermined value. It is preferable such value is set appropriately for each target person, including whether obesity exists in the target person. For example, the value can be set to about 5% or 10%, and it is preferable to set the value to less than 5% from the viewpoint of performing extraction of “the unbalanced nutrition” with a high accuracy. Further, in Step S24, when the value of the C-D (%) is equal to or greater than the predetermined value (i.e., YES) the procedure proceeds to Step S25. In this case, the factor, “the unbalanced nutrition” by the dinner, is extracted. Alternatively, in Step S24, when the value of the C-D (%) is less than the predetermined value (i.e., NO), the flow is terminated. The present embodiment's lifestyle improvement support system is configured to also perform the same flow for breakfast and lunch.

In the above description, “the unbalanced nutrition” at each of breakfast, lunch, and dinner is set as the factor candidate. Note, the present embodiment's lifestyle improvement support system is not limited to a single meal. For example, “the unbalanced nutrition in the whole day” including all the unbalanced nutrition calculations for the target person's breakfast, lunch, and dinner can be set as the factor candidate.

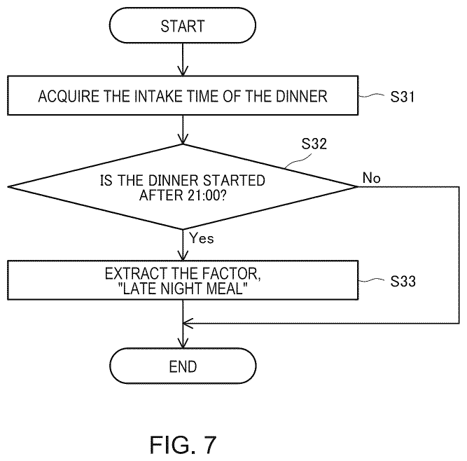

Next, an example of a flowchart indicating “the late night meal” among the candidate factors is extracted as a factor is described. FIG. 7 is a flowchart illustrating a procedure whereby “the late-night meal” is extracted as a factor. As shown in FIG. 7, first, in Step S31, the intake time of the target person's dinner is acquired. Next, in Step S32, it is determined whether the target person started their dinner after 21:00. When it is determined in Step S32 the target person started their dinner after 21:00 (i.e., YES), the procedure proceeds to Step S33. In this case, the factor, “the late night meal” for the target person's dinner, is extracted. Alternatively, in Step S32, when it is determined the target person started the dinner before 21:00 (i.e., NO), the flow is terminated. In the above description, although the reference time is set to 21:00, the present embodiment's lifestyle improvement support system is not limited to this specific time, and the reference time can be changed as appropriate. For example, the reference time may be set to 19:00 or 22:00.

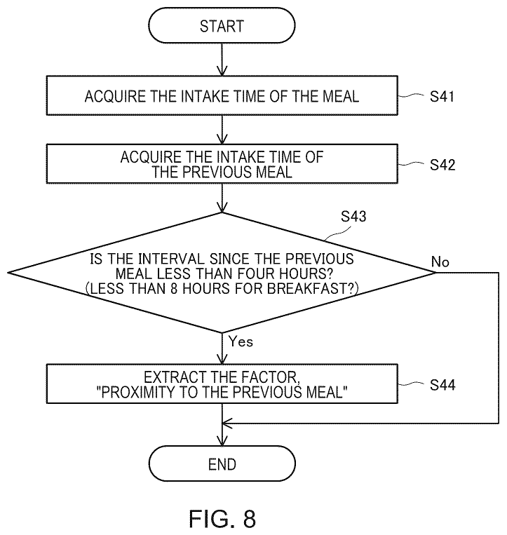

Next, an example of a flowchart indicating “the time gap with a previous meal” among the candidate factors is extracted as a factor is described. FIG. 8 is a flowchart illustrating a procedure whereby “the time gap with the previous meal” is extracted as a factor. The flow is described when breakfast is used as an example of the meals. The present embodiment's lifestyle improvement support system is configured to also perform the same flow for lunch and dinner. As shown in FIG. 8, first, in Step S41, the intake time of the target person's breakfast is acquired. Next, in Step S42, the previous day's intake time of the target person's dinner, which is the meal preceding the target person's breakfast, is acquired. In this case, the meal preceding the target person's dinner corresponds to their lunch on the same day, and meal preceding the target person's lunch corresponds to their breakfast on the same day (the same applies hereinafter). Next, in Step S43, it is determined whether the interval between the intake times of the target person's breakfast and their previous day's dinner is shorter than a predetermined interval. For example, in the case of breakfast and lunch, or lunch and dinner, the interval can be set to be shorter than about 4 hours. For example, in the case of breakfast and the target person's previous day's dinner, the interval can be set to be shorter than about 8 hours. Note, the interval may be changed as appropriate. Further, in Step S43, when the interval between the intake times of the target person's breakfast and their previous day's dinner is shorter than 8 hours (i.e., YES), the procedure proceeds to step S44. In this case, the factor, “the time gap with the previous meal” for the target person's breakfast, is extracted. Alternatively, in Step S43, the interval between the intake times for the target person's breakfast and their previous day's dinner is equal to or greater than 8 hours (i.e., NO), the flow is terminated. The present embodiment's lifestyle improvement support system is configured to also perform the same flow for lunch and dinner.

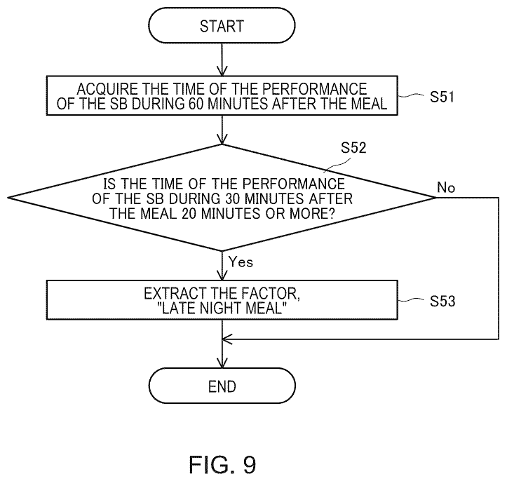

Next, an example of a flowchart indicating “the excessive performance of SB during 30 minutes after a meal” among the candidate factors is extracted as a factor is described. FIG. 9 is a flowchart illustrating a procedure whereby “the excessive performance of SB during 30 minutes after a meal” is extracted as a factor. The flow is described when breakfast is used as an example of the meals. The present embodiment's lifestyle improvement support system is configured to also perform the same flow for lunch and dinner. As shown in FIG. 9, first, in Step S51, the target person's performance time of SB (i.e., physical activity of low intensity) during 60 minutes after the target person's breakfast is acquired. Next, in Step S52, it is determined whether the target person's performance time of SB during 30 minutes after the target person's breakfast is 20 minutes or more. Next, in Step S52, when the target person's performance time is 20 minutes or more (i.e., YES), the procedure proceeds to Step S53. In this case, the factor, “the performance of SB during 30 minutes after the meal,” is extracted. Alternatively, in Step S52, the performance time of SB during 30 minutes after the target person's breakfast is less than 20 minutes (i.e., NO), the flow ends. The present embodiment's lifestyle improvement support system is configured to also perform the same flow for lunch and dinner. In the above description, although it is determined using 30 minutes after breakfast as the reference time for performing SB, the present embodiment's lifestyle improvement support system is not limited to this time, and the reference time can be changed as appropriate. For example, such reference time may be 30 to 60 minutes after the target person's breakfast.

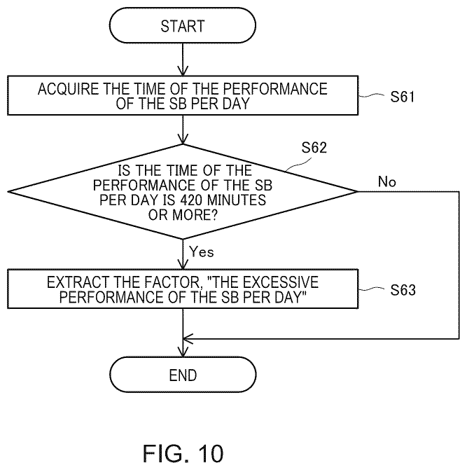

Next, an example of a flowchart indicating “the excessive performance of SB in a day” among the candidate factors is extracted as a factor is described. FIG. 10 is a flowchart illustrating a procedure whereby “the excessive performance of SB in a day” is extracted as a factor. As shown in FIG. 10, first, in Step S61, the time of the target person's performance of SB (i.e., physical activity of low intensity) in a day is acquired. Next, in Step S62, it is determined whether the target person's time for performing SB in a day is 420 minutes or more. Next, in Step S62, when the target person's time for performing SB is 420 minutes or more (i.e., YES), the procedure proceeds to Step S63. In this case, the factor, “the excessive performance of SB in a day,” is extracted. Alternatively, in Step S62, the performance time of SB in a day is less than 420 minutes (i.e., NO), the flow ends. In the above description, although it is determined using 420 minutes in a day as the reference time for performing SB, the present embodiment's lifestyle improvement support system is not limited to this specific time, and the reference time can be changed as appropriate. For example, the reference time may be set to 300 minutes or 500 minutes.

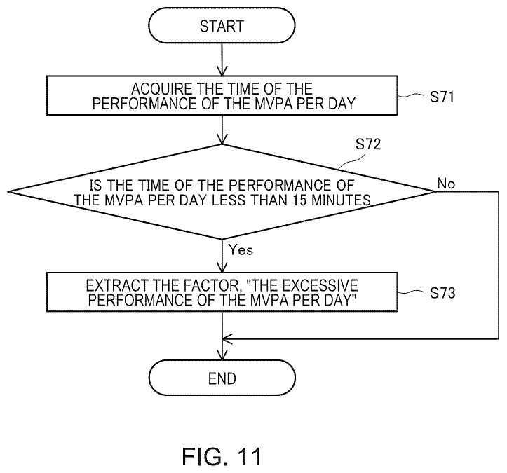

Next, an example of a flowchart indicating “the insufficient performance of MVPA in a day” among the candidate factors is extracted as a factor is described. FIG. 11 is a flowchart illustrating a procedure whereby “the insufficient performance of MVPA in a day” is extracted as a factor. As shown in FIG. 11, first, in Step S71, the time for the target person performing MVPA (i.e., physical activity of moderate intensity) in a day is acquired. Next, in Step S72, it is determined whether the time for the target person performing MVPA in a day is less than 15 minutes. Further, in Step S72, when the time for the target person performing MVPA is less than 15 minutes (i.e., YES), the procedure proceeds to Step S73. In this case, the factor, “the excessive performance of MVPA in a day,” is extracted. Alternatively, in Step S72, the time for the target person performing MVPA in a day is 15 minutes or more (i.e., NO), the flow ends. In the above description, although it is determined using 15 minutes as the reference time for performing MVPA in a day, the present embodiment's lifestyle improvement support system is not limited to this specific time, and the reference time can be changed as appropriate. For example, the reference time may be set to 10 minutes or 30 minutes.

As described above, the present embodiment's lifestyle improvement support system is configured to execute the flow for each meal (i.e., breakfast, lunch, and dinner) related to “the excessive intake of calories,” “the unbalanced nutrition,” “the time gap with the previous meal,” and “the excessive performance of SB during 30 minutes after the meal.” In such cases, the factors can be extracted as follows. Specifically, for example, in the case where “the excessive intake of calories” is extracted as the factor only at lunch regarding the flow related to “the excessive intake of calories,” “the excessive intake of calories” at lunch can be extracted as the factor. As another example, in the case where “the unbalanced nutrition” is extracted as the factor at lunch and dinner regarding the flow related to “the unbalanced nutrition”, “the unbalanced nutrition” at lunch and dinner can be extracted as the factor. Other factors can be similarly extracted.

As for the target person A, the flow of each factor described above is executed based on the target person A's physical activity data and dietary activity data. As a result, six factors are extracted (specifically, “the excessive intake of calories” (more specifically, the excessive intake of calories at dinner), “the unbalanced nutrition” (more specifically, unbalanced carbohydrate at dinner), “the time gap with the previous meal” (more specifically, the time gap between breakfast and lunch), “the excessive performance of SB during 30 minutes after a meal” (more specifically, excessive performance of SB during the 30 minutes after dinner), “the excessive performance of SB in a day”, and “the insufficient performance of MVPA in a day”). Regarding the above six factors, “the excessive intake of calories” and “the unbalanced nutrition” can be classified into categories related to meal content, and “the time gap with a previous meal” can be classified into categories related to dietary behavior. Further, “the excessive performance of SB during 30 minutes after a meal” can be classified into categories related to physical activity within a given time, and “the excessive performance of SB in a day” and “the insufficient performance of MVPA in a day” can be classified into categories related to physical activity in a day. That is, these six factors can be classified into four categories. Further, as a result of further considering the target person A'S health data, it is determined whether the target person A can improve their lifestyle in relation to the above six factors, and therefore, it is assumed the six factors are extracted. The six factors are stored in the Storage unit 22.

Next, in Step S3 of FIG. 4, two or more factors are selected from among the factors extracted in step S2. Specifically, the selection unit 25 notifies the target person A about the six factors extracted in step S2, acquires the target person A's self-efficacy for improving their lifestyle in relation to the six factors, and selects the factors in consideration of the target person A's self-efficacy. First, the selection unit 25 displays the six factors stored in the storage unit 22 on the display 11 of the target person A's terminal 10 through the communication unit 23. Next, the target person A inputs their self-efficacy for improving their lifestyle in relation to the six factors, whereby information on the target person A's self-efficacy is acquired from the input unit. Next, the self-efficacy information is transmitted from the terminal controlling device 13 to the communication unit 23, and the information is stored in the storage unit 22. In this case, since the target person A's self-efficacy for their lifestyle improvement was high in relation to the six factors, the system decides to select a factor from among the six factors. In addition, the selection unit 25 is configured to select the factors based on a selection criterion (or a program having the selection criterion), which is a criterion prompting the target person A to perform the same lifestyle changes for one more day, when increasing the number of the categories by one day each and selecting the factors from among the categories (including the increased categories), or when it cannot be confirmed the target person's blood glucose level improved based on the target person improving their lifestyle. In Step S3, the selection unit 25 selects the category related to the target person's meal content (i.e., two factors of “the excessive intake of calories” and “the unbalanced nutrition”).

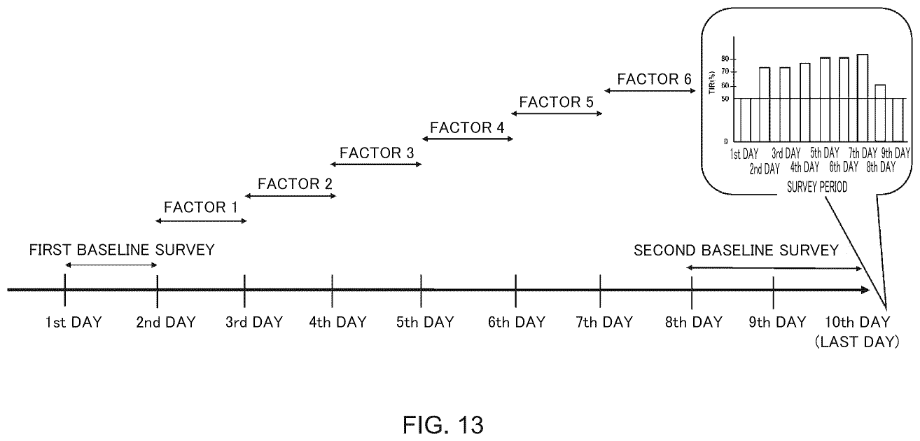

The factors (1) to (6) in FIG. 3 correspond to “the excessive intake of calories,” “the unbalanced nutrition,” “the time gap with a previous meal,” “the excessive performance of SB during 30 minutes after a meal,” “the excessive performance of SB in a day,” and “the insufficient performance of MVPA in a day,” respectively. In FIG. 3, the types of factors are shown for convenience, and it means the target person improving their lifestyle in relation to the specified factors. The factors of FIG. 13 described later are the same as the factors of FIG. 3.

Next, the second day in FIG. 3 is described with reference to FIG. 4. In Step S4 of FIG. 4, the two factors selected in step S3 are notified to the target person, and improving the target person's lifestyle in relation to the two factors is stimulated. Specifically, the first notification unit 26 notifies the target person about improving the target person's lifestyle in relation to the two factors selected in Step S3 (for example, “reduction of the caloric intake at dinner to predetermined calories or less” and “reduction of the ratio of carbohydrate intake at dinner to a predetermined value or lower”) in order to stimulate improving the target person's lifestyle in relation to these two factors. The specific means of the notification by the first notification unit 26 is not particularly limited. For example, the first notification unit 26 may display the corresponding message on the display 11 (see FIG. 2) of the target person's terminal 10. Alternatively, the first notification unit 26 may transmit the corresponding email to the target person's terminal 10. The second notification unit 28 described later is the same as the first notification unit 26. The target person A improves their lifestyle in accordance with the notification from the first notification unit 26.

Next, in Step S5 of FIG. 4, the target person A's blood glucose level data (TIR (%) in this case) after the improvement of the target person's lifestyle in relation to the two factors is acquired. Specifically, the acquisition device 12 acquires the target person A's blood glucose level data transmitted from the continuous blood glucose meter 30 by wireless communication. The terminal controlling device 13 transmits the blood glucose level data acquired by the acquisition device 12 to the management server 20, and the blood glucose level data is stored in the storage unit 22.

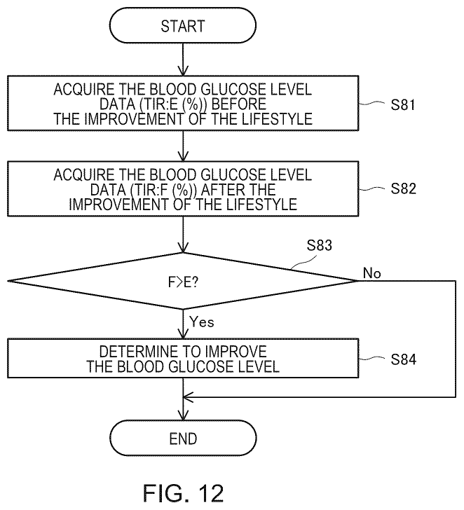

Next, in Step S6 of FIG. 4, it is determined whether improving the target person A's lifestyle contributed to improving their blood glucose level. Specifically, the determination unit 27 determines whether the target person A's improved factor contributed to improving their blood glucose level, based on the blood glucose level data acquired from the target person A before the improvement of their lifestyle and the blood glucose level data acquired from the target person A after the improvement of their lifestyle. FIG. 12 is a flowchart illustrating a procedure for determining the target person's blood glucose level is improved according to the present embodiment. First, in Step S81, the target person's blood glucose level data (TIR:E (%)) before the improvement of the target person's lifestyle is acquired. Specifically, the determination unit 27 acquires the blood glucose level data related to the first baseline survey stored in the storage unit 22. Next, in Step S82, the target person's blood glucose level data (TIR:F (%)) after the improvement of the target person's lifestyle is acquired. Specifically, the determination unit 27 acquires the blood glucose level data of the second day stored in the storage unit 22. Next, in Step S83, it is determined whether F>E. Further, in Step S83, when it is determined F>E (i.e., YES), the procedure proceeds to Step S84. In this case, it is determined the blood glucose level is improved. Alternatively, in Step S83, when it is determined F≤E (i.e., NO), the flow is terminated. In this manner, it is determined whether the target person's blood glucose level improved. Note, the two factors, “the excessive intake of calories” and “the unbalanced nutrition,” were determined to have contributed to improving the target person A's blood glucose levels. The result of the determination of F>E is stored in the storage unit 22.

Next, in Step S7, it is determined whether the predetermined program is completed in the selection unit 25. Note, when it is determined Steps S3 to S6 are not executed for the remaining three categories, the procedure returns to Step S3. In Step S3, the selection unit 25 selects the category related to the target person's meal content (i.e., two factors of “the excessive intake of calories” and “the unbalanced nutrition”), and the category related to the target person's dietary behavior (i.e., “the time gap with the previous meal”), from among the factors extracted in step S2.

Next, the third day in FIG. 3 is described with reference to FIG. 4. First, in Step S4 of FIG. 4, the three factors selected in Step S3 are notified to the target person, and the improvement of the target person's lifestyle in relation to these three factors is stimulated. Specifically, the first notification unit 26 notifies the target person about the improvement of the target person's lifestyle in relation to the three factors selected in Step S3 (for example, “the reduction of the caloric intake at dinner to predetermined calories or less” and “the reduction of the ratio of the carbohydrate intake at dinner to a predetermined value or lower” and “the interval between breakfast and lunch set to be equal to or more than 4 hours”) in order to stimulate the improvement of the target person's lifestyle in relation to the three factors selected in Step S3. Next, in Step S5, the acquisition device 12 acquires the target person A's blood glucose level data after the improvement of the target person's lifestyle in relation to the above three factors. In Step S6, the determination unit 27 determines whether a target person A's improved factor contributed to improving the target person A's blood glucose level, based on the blood glucose level data acquired from the target person A before the improvement of the target person A's lifestyle (i.e., on the second day) and the blood glucose level data acquired from the target person A after the improvement of their lifestyle (i.e., on the third day). As a result, it is determined whether improving the target person A's lifestyle in relation to “the time gap with the previous meal” contributed to improving the target person A's blood glucose level. The result of the determination concerning improving the target person A's lifestyle is stored in the storage unit 22. Next, in Step S7, it is determined whether the predetermined program is completed in the selection unit 25. Note, when it is determined Steps S3 to S6 are not executed for the remaining two categories, the procedure returns to Step S3. In Step S3, the selection unit 25 selects the category related to the target person's meal content (i.e., two factors of “the excessive intake of calories” and “the unbalanced nutrition”), the category related to the target person's dietary behavior (i.e., “the time gap with the previous meal”), and the category related to the target person's physical activity within a predetermined time (i.e., “the excessive performance of SB during 30 minutes after a meal”), from among the factors extracted in step S2.

Next, the fourth day in FIG. 3 is described with reference to FIG. 4. First, in Step S4 of FIG. 4, the four factors selected in Step S3 are notified to the target person, and the improvement of the target person's lifestyle in relation to the four factors is stimulated. Specifically, the first notification unit 26 notifies the target person about the improvement of the target person's lifestyle in relation to the four factors selected in Step S3 (for example, “the reduction of the caloric intake at dinner to predetermined calories or less” and “the reduction of the ratio of the carbohydrate intake at dinner to a predetermined value or lower,” “the time gap between breakfast and lunch set to be equal to 4 hours or more,” and “the performance time of SB during 30 minutes after dinner is less than 20 minutes”) in order to stimulate improving the target person's lifestyle in relation to the four factors selected in Step S3. Next, in Step S5, the acquisition device 12 acquires the target person A's blood glucose level data after the improvement of their lifestyle in relation to the above-specified four factors. In Step S6, the determination unit 27 determines whether a target person A's improved factor contributed to improving their blood glucose level, based on the blood glucose level data acquired from the target person A before the improvement of their lifestyle (i.e., on the third day) and the blood glucose level data acquired from the target person A after the improvement of their lifestyle (i.e., on the fourth day). As a result, it is determined whether improving the target person A's lifestyle in relation to “the excessive performance of SB during 30 minutes after a meal” contributed to improving the target person A's blood glucose level. The result of the determination concerning improving the target person A's lifestyle is stored in the storage unit 22. Next, in Step S7, it is determined whether the predetermined program is completed in the selection unit 25. Note, when it is determined Steps S3 to S6 are not executed for the single remaining category, the procedure returns to Step S3. In Step S3, the selection unit 25 selects the category related to the target person's meal content (i.e., two factors of “the excessive intake of calories” and “the unbalanced nutrition”), the category related to the target person's dietary behavior (i.e., “the time gap with the previous meal”), the category related to the target person's physical activity within a predetermined time (i.e., “the excessive performance of SB during 30 minutes after a meal”), and the category related to the target person's physical activity in a day (i.e., two factors of “the excessive performance of SB in a day” and “the insufficient performance of MVPA in a day”), from among the factors extracted in step S2.

Next, the fifth day in FIG. 3 is described with reference to FIG. 4. First, in Step S4 of FIG. 4, the six factors selected in Step S3 are notified to the target person, and improving the target person's lifestyle in relation to the six factors is stimulated. Specifically, the first notification unit 26 notifies the target person about the improvement of the target person's lifestyle in relation to the six factors selected in Step S3 (for example, “the reduction of the caloric intake at dinner to predetermined calories or less” and “the reduction of the ratio of the carbohydrate intake at dinner to a predetermined value or lower,” “time gap interval between breakfast and lunch set to be equal to or more than 4 hours,” “the performance time of SB during 30 minutes after dinner is less than 20 minutes,” “the performance time of SB in a day set to be less than 420 minutes,” and “the performance time of MVPA in a day set to be 15 minutes or more”) in order to stimulate improving the target person A's lifestyle in relation to the six factors selected in Step S3. Next, in Step S5, the acquisition device 12 acquires the target person A's blood glucose level data after the improvement of their lifestyle in relation to the above-specified six factors. In Step S6, the determination unit 27 determines whether a target person A's improved factor contributed to improving the target person A's blood glucose level, based on the blood glucose level data acquired from the target person A before the improvement of their lifestyle (i.e., on the fourth day) and the blood glucose level data acquired from the target person A after the improvement of their lifestyle (i.e., on the fifth day). As a result, it is determined whether improving the target person A's lifestyle in relation to “the excessive performance of SB in a day” and “the insufficient performance of MVPA in a day” did not contribute to improving the target person A's blood glucose level. The result of the determination concerning improving the target person A's lifestyle is stored in the storage unit 22. Next, in Step S7, it is determined whether the predetermined program is completed in the selection unit 25. Note, when it is determined Steps S3 to S6 are not executed again for the combination of the factors (i.e., the combination of the six factors), they are determined not to have contributed to improving the target person's blood glucose level, and the procedure returns to step S3. In Step S3, the selection unit 25 re-selects the category related to the target person's meal content (i.e., two factors of “the excessive intake of calories” and “the unbalanced nutrition”), the category related to the target person's dietary behavior (i.e., “time gap with the previous meal”), the category related to the target person's physical activity within a predetermined time (i.e., “excessive performance of SB during 30 minutes after a meal”), and the category related to the target person's physical activity in a day (i.e., two factors of “excessive performance of SB in a day” and “insufficient performance of MVPA in a day”), from among the factors extracted in step S2.

Next, the sixth day in FIG. 3 is described with reference to FIG. 4. First, in Step S4 of FIG. 4, the six factors selected in Step S3 are notified to the target person, and improving the target person's lifestyle in relation to the six factors is stimulated. Specifically, the first notification unit 26 notifies the target person about the improvement of the target person's lifestyle in relation to the six factors in order to stimulate improving the target person's lifestyle in relation to the six factors in Step 3. Next, in Step S5, the acquisition device 12 acquires the target person A's blood glucose level data after the improvement of their lifestyle in relation to the above six factors. In Step S6, the determination unit 27 determines whether a target person A's improved factor contributed to improving their blood glucose level, based on the blood glucose level data acquired from the target person A before the improvement of their lifestyle (i.e., on the fifth day) and the blood glucose level data acquired from the target person A after the improvement of their lifestyle (i.e., on the sixth day). As a result, it is determined whether improving the target person A's lifestyle in relation to “the excessive performance of SB in a day” and “the insufficient performance of MVPA in a day” contributed to improving the target person A's blood glucose level. The result of the determination concerning improving the target person A's lifestyle is stored in the storage unit 22. Next, in Step S7, it is determined whether the predetermined program is completed in the selection unit 25. Note, when it is determined the program is completed, the procedure proceeds to step S8.

Next, the seventh and eighth days in FIG. 3 are described with reference to FIG. 4. In Step S8 of FIG. 4, the data related to the second baseline survey about the target person A (for example, blood glucose level data) is acquired and is checked. Specifically, it is checked whether the blood glucose level data (TIR (%)) acquired in the second baseline survey approaches (or returns to) the blood glucose level data (TIR (%)) acquired in the first baseline survey. More specifically, the acquisition device 12 acquires the target person A's blood glucose level data transmitted by wireless communication from the continuous blood glucose meter 30 attached to the target person A. The terminal controlling device 13 transmits the blood glucose level data acquired by the acquisition device 12 to the management server 20, and the blood glucose level data is stored in the storage unit 22. When the blood glucose level data (TIR (%)) of the second baseline survey is recognized to approach (or return to) the blood glucose level data (TIR (%)) of the first baseline survey, it is preferable to perform the lifestyle improvement support method according to the above procedure, since improving the target person's lifestyle contributed to improving blood glucose level can be indicated as evidence.

The bar graph of FIG. 3 collectively shows the transition of the target person A's blood glucose level data (TIR (%)) from the first day to the eighth day described above. It is confirmed the blood glucose level data (TIR (%)) related to the second baseline survey is returned to the blood glucose level data (TIR (%)) related to the first baseline survey based on the results of the seventh and eighth days of the bar graph of FIG. 3. Thus, it can be confirmed that improving the target person A's lifestyle contributes to improving the target person's blood glucose level.

Next, the ninth day in FIG. 3 is described with reference to FIG. 4. In Step S9 of FIG. 4, the factor to be improved is notified to the target person A. Specifically, the second notification unit 28 notifies the target person A about one or more of the factors from among the factors that are determined to have contributed to improving the target person A's blood glucose level, as the factor to be improved, in order to stimulate improving the target person A's lifestyle in relation to the specified factor, based on the results from the first day to the eighth day stored in the storage unit 22. In this case, the second notification unit 28 notifies the target person about the six factors as the factors to be improved.

As described above, according to the first lifestyle improvement support method, the factors to be improved can be proposed to the target person. According to this method, the factors to be improved can be proposed to the target person based on whether the target person's blood glucose level data is improved (i.e., based on the target person's specific data). Therefore, the first lifestyle improvement support method can appropriately support improving the target person's lifestyle.

### <Second Lifestyle Improvement Support Method>

Next, the second lifestyle improvement support method is described. FIG. 13 is a diagram explaining the second lifestyle improvement support method using the lifestyle improvement support system according to the present embodiment. FIG. 14 is a flowchart illustrating a procedure of the second lifestyle improvement support method using the lifestyle improvement support system according to the present embodiment. The second lifestyle improvement support method is described below with reference to the drawings as needed. Note, the second lifestyle improvement support method is performed by a target person B, as a target person. Further, during the period from the first day to the tenth day in FIG. 13, the target person B has the sensor attached to the continuous blood glucose meter 30 on the target person B's upper arm and has the sensor device 40 on the target person B's wrist (see FIG. 1).

First, the first day in FIG. 13 is described with reference to FIG. 14. Initially, in Step S91 of FIG. 14, the data related to a first baseline survey about the target person is acquired. For example, the target person B's blood glucose level data, physical activity data, dietary activity data, and health data are acquired in the first baseline survey. Specifically, the acquisition device 12 of FIG. 2 acquires the target person B's blood glucose level data and physical activity data from the continuous blood glucose meter 30 and the sensor device 40 attached to the target person B, respectively. The acquisition device 12 also acquires the target person B's dietary activity data and health data input by the target person B. It is preferable the dietary activity data of the day before the first baseline survey is also acquired to use the dietary activity data when extracting the factors described later. The terminal controlling device 13 transmits the various data acquired by the acquisition device 12 to the communication unit 23, and the various data are stored in the storage unit 22.

Next, in Step S92 of FIG. 14, a plurality of factors for the target person is extracted. Specifically, the extraction unit 24 extracts the plurality of factors related to the target person's physical activity and dietary activity to be improved by the target person B, based on the various data acquired in Step S91 (for example, blood glucose level data, physical activity data, dietary activity data, and health data). In the present embodiment, as in the first lifestyle improvement support method described above, the factors are extracted from among seven candidate factors (specifically, “the excessive intake of calories,” “the unbalanced nutrition,” “the time gap with a previous meal,” “the late night meal,” “the excessive performance of SB during 30 minutes after a meal,” “the excessive performance of SB in a day,” and “the insufficient performance of MVPA in a day”) that are assumed by the present inventor to be easily involved in the fluctuation of blood glucose level.

As for target person B, the flow of each factor as described in the first lifestyle improvement support method is executed based on the target person's physical activity data and dietary activity data. As a result, six factors are extracted (specifically, “the excessive intake of calories” (more specifically, the excessive intake of calories at dinner), “the unbalanced nutrition” (more specifically, the excessive intake rate of carbohydrates at dinner), “the time gap with the previous meal” (more specifically, the time gap between breakfast and lunch), “the excessive performance of SB during 30 minutes after a meal” (more specifically, the excessive performance of SB during 30 minutes after dinner), “the excessive performance of SB in a day”, and “the insufficient performance of MVPA in a day”). Further, as a result of further considering the target person7s health data, it is determined whether the target person B can improve their lifestyle in relation to the above-specified six factors, and therefore, it is assumed the six factors are extracted. The six factors are stored in the storage unit 22.

Next, in Step S93 of FIG. 14, one of the factors is selected from the factors extracted in step S92. Specifically, the selection unit 25 notifies the target person B about the six factors extracted in step S92, acquires the target person B's self-efficacy for improving their lifestyle in relation to the six factors, and selects the factors in consideration of the self-efficacy. First, the selection unit 25 displays the six factors stored in the storage unit 22 on the display 11 of the target person's terminal 10 through the communication unit 23. Next, the target person B inputs the self-efficacy for improving their lifestyle in relation to the six factors, whereby information on the target person B's self-efficacy is acquired from the input unit. Next, the information on self-efficacy is transmitted from the terminal controlling device 13 to the communication unit 23, and the information is stored in the storage unit 22. In this case, since the target person B's self-efficacy for their lifestyle improvement was high in relation to the six factors, it was decided to select a factor from among the six factors. In addition, the selection unit 25 is configured to select the factors, based on a selection criterion (or a program having the selection criterion), such as selecting one of the above six factors each day. In Step S93, the selection unit 25 selects “the excessive intake of calories.”

Next, the second day in FIG. 13 is described with reference to FIG. 14. In Step S94 of FIG. 14, the factor selected in step S93 is notified to the target person, and improving the target person's lifestyle in relation to the factor is stimulated. Specifically, the first notification unit 26 notifies the target person B about the improvement of their lifestyle in relation to the factor selected in Step 93 (i.e., “reduction of the caloric intake at dinner to a predetermined calorific value or lower”) in order to stimulate improving the target person B's lifestyle in relation to the specified factor. The target person B improves their lifestyle in accordance with the notification from the first notification unit 26.

Next, in Step S95 of FIG. 14, the target person B's blood glucose level data (TIR (%) in this case) after the improvement of their lifestyle in relation to the factor is acquired. Specifically, the acquisition device 12 acquires the target person B's blood glucose level data transmitted by wireless communication from the target person B's continuous blood glucose meter 30. The terminal controlling device 13 transmits the blood glucose level data acquired by the acquisition device 12 to the management server 20, and the blood glucose level data is stored in the storage unit 22.

Next, in Step S96 of FIG. 14, it is determined whether the predetermined program is completed in the selection unit 25. Note, when it is determined Steps S93 to S95 are not executed for the remaining five factors, the procedure returns to Step S93. In Step S93, the selection unit 25 selects “the unbalanced nutrition.”

Next, the third day in FIG. 13 is described with reference to FIG. 14. In Step S94 of FIG. 14, the factor selected in step S93 is notified to the target person B, and improving their lifestyle in relation to the factor is stimulated. Specifically, the first notification unit 26 notifies the target person B about the improvement of their lifestyle in relation to the factor selected in Step 93 (i.e., “the ratio of the carbohydrate intake at dinner is set to a predetermined value or lower”) in order to stimulate improving the target person B's lifestyle in relation to the specified factor. The target person B thus improves their lifestyle in accordance with the notification from the first notification unit 26.

Next, in Step S95 of FIG. 14, the target person B's blood glucose level data after the improvement of the target person B's lifestyle in relation to the above-specified factor is acquired. Specifically, the acquisition device 12 acquires the target person B's blood glucose level data transmitted by wireless communication from the target person B's continuous blood glucose meter 30. The terminal controlling device 13 transmits the blood glucose level data acquired by the acquisition device 12 to the management server 20, and the blood glucose level data is stored in the storage unit 22.

Next, in Step S96 of FIG. 14, it is determined whether the predetermined program is completed in the selection unit 25. Note, when it is determined Steps S93 to S95 are not executed for the remaining four factors, the procedure returns to Step S93. In Step S93, the selection unit 25 selects “the time gap with a previous meal.”

Next, the fourth day in FIG. 13 is described with reference to FIG. 14. In Step S94 of FIG. 14, the factor selected in step S93 is notified to the target person B, and improving the target person B's lifestyle in relation to the factor is stimulated. Specifically, the first notification unit 26 notifies the target person B about the improvement of the target person B's lifestyle in relation to the factor selected in Step 93 (i.e., “the interval between breakfast and lunch set to be equal to or more than 4 hours”) in order to stimulate improving the target person B's lifestyle in relation to the specified factor. The target person B thus improves their lifestyle in accordance with the notification from the first notification unit 26.

Next, in Step S95 of FIG. 14, the target person B's blood glucose level data after the improvement of their lifestyle in relation to the above-specified factor is acquired. Specifically, the acquisition device 12 acquires the target person B's blood glucose level data transmitted by wireless communication from the target person B's continuous blood glucose meter 30. The terminal controlling device 13 transmits the blood glucose level data acquired by the acquisition device 12 to the management server 20, and the blood glucose level data is stored in the storage unit 22.

Next, in Step S96 of FIG. 14, it is determined whether the predetermined program is completed in the selection unit 25. Note, when it is determined Steps S93 to S95 are not executed for the remaining three factors, the procedure returns to Step S93. In Step S93, the selection unit 25 selects “excessive performance of SB during 30 minutes after a meal.”

Next, the fifth day in FIG. 13 is described with reference to FIG. 14. In Step S94 of FIG. 14, the factor selected in step S93 is notified to the target person B, and improving their lifestyle in relation to the factor is stimulated. Specifically, the first notification unit 26 notifies the target person B about the improvement of the target person B's lifestyle in relation to the factor selected in Step 93 (i.e., “the performance time of SB during 30 minutes after dinner is less than 20 minutes”) in order to stimulate improving the target person B's lifestyle in relation to the specified factor. The target person B thus improves their lifestyle in accordance with the notification from the first notification unit 26.

Next, in Step S95 of FIG. 14, the target person B's blood glucose level data after the improvement of their lifestyle in relation to the above-specified factor is acquired. Specifically, the acquisition device 12 acquires the target person B's blood glucose level data transmitted by wireless communication from the target person B's continuous blood glucose meter 30. The terminal controlling device 13 transmits the blood glucose level data acquired by the acquisition device 12 to the management server 20, and the blood glucose level data is stored in the storage unit 22.

Next, in Step S96 of FIG. 14, it is determined whether the predetermined program is completed in the selection unit 25. Note, when it is determined Steps S93 to S95 are not executed for the remaining two factors, the procedure returns to Step S93. In Step S93, the selection unit 25 selects “the excessive performance of SB in a day.”

Next, the sixth day in FIG. 13 is described with reference to FIG. 14. In Step S94 of FIG. 14, the factor selected in step S93 is notified to the target person B, and improving their lifestyle in relation to the factor is stimulated. Specifically, the first notification unit 26 notifies the target person B about the improvement of their lifestyle in relation to the factor selected in Step 93 (i.e., “the performance time of SB in a day set to be less than 420 minutes”) in order to stimulate improving the target person B's lifestyle in relation to the specified factor. The target person B thus improves their lifestyle in accordance with the notification from the first notification unit 26.

Next, in Step S95 of FIG. 14, the target person B's blood glucose level data after the improvement of their lifestyle in relation to the above-specified factor is acquired. Specifically, the acquisition device 12 acquires the target person B's blood glucose level data transmitted by wireless communication from the target person B's continuous blood glucose meter 30. The terminal controlling device 13 transmits the blood glucose level data acquired by the acquisition device 12 to the management server 20, and the blood glucose level data is stored in the storage unit 22.

Next, in Step S96 of FIG. 14, it is determined whether the predetermined program is completed in the selection unit 25. Note, when it is determined Steps S93 to S95 are not executed for the one remaining factor, the procedure returns to Step S93. In Step S93, the selection unit 25 selects “excessive performance of SB in a day.”

Next, the seventh day in FIG. 13 is described with reference to FIG. 14. In Step S94 of FIG. 14, the factor selected in step S93 is notified to the target person B, and improving their lifestyle in relation to the specified factor is stimulated. Specifically, the first notification unit 26 notifies the target person B about the improvement of their lifestyle in relation to the factor selected in Step S93 (i.e., “the performance time of MVPA in a day set to be less than 15 minutes”) in order to stimulate improving the target person B's lifestyle in relation to the specified factor. The target person B thus improves their lifestyle in accordance with the notification from the first notification unit 26.

Next, in Step S95 of FIG. 14, the target person B's blood glucose level data after the improvement of their lifestyle in relation to the above-specified factor is acquired. Specifically, the acquisition device 12 acquires the target person B's blood glucose level data transmitted by wireless communication from the target person B's continuous blood glucose meter 30. The terminal controlling device 13 transmits the blood glucose level data acquired by the acquisition device 12 to the management server 20, and the blood glucose level data is stored in the storage unit 22.

Next, in Step S96 of FIG. 14, it is determined whether the predetermined program is completed in the selection unit 25. Note, when it is determined the program is completed, the procedure proceeds to step S97.

Next, in Step S97 of FIG. 14, it is determined whether each of the improvements in the target person B's lifestyle in relation to the six factors contributed to improving the target person B's blood glucose level. Specifically, the determination unit 27 determines whether a target person B's improved factor contributed to improving their blood glucose level, based on the blood glucose level data acquired from the target person B before the improvement of their lifestyle and the blood glucose level data acquired from the target person B after the improvement of their lifestyle. More specifically, refer to FIG. 12 and the description of step S6 in the first lifestyle improvement support method. When performed collectively the above-described determination based on the blood glucose level data related to the first baseline survey and the blood glucose level data related to the first day to seventh day stored in the storage unit 22, the determination unit 27 determines whether improving the target person's lifestyle in relation to the four factors (specifically, “excessive intake of calories,” “time gap with a previous meal,” “excessive performance of SB during 30 minutes after a meal,” and “insufficient performance of MVPA in a day”) contributed to improving the target person B's blood glucose level.

Next, the eighth and ninth days in FIG. 13 are described with reference to FIG. 14. In Step S98 of FIG. 14, the data related to the second baseline survey about the target person B is acquired and is checked. Specifically, the acquisition device 12 acquires the blood glucose level data transmitted from the continuous blood glucose meter 30 attached to the target person B. The terminal controlling device 13 transmits the various data acquired by the acquisition device 12 to the communication unit 20, and the various data is stored in the storage unit 22. Specifically, it is checked whether the blood glucose level data (TIR (%)) acquired in the second baseline survey approaches (or returns to) the blood glucose level data (TIR (%)) acquired in the first baseline survey. When the blood glucose level data (TIR (%)) of the second baseline survey is recognized to approach (or return to) the blood glucose level data (TIR (%)) of the first baseline survey, it is preferable to perform the lifestyle improvement support method according to the above-described procedure, since improving the target person's lifestyle contributed to improving their blood glucose level can be indicated as evidence. For example, it is assumed such deterioration is confirmed.

FIG. 13's bar graph collectively shows the transition of the above-described target person B's blood glucose level data (TIR (%)) from the first day to the ninth day as described above. It is confirmed the blood glucose level data (TIR (%)) related to the second baseline survey returned to the blood glucose level data (TIR (%)) related to the first baseline survey, based on the results of the eighth and ninth days of FIG. 13's bar graph, and thus it can be confirmed that improving the target person B's lifestyle contributes to improving their blood glucose level.

Next, the tenth day in FIG. 13 is described with reference to FIG. 14. In Step S99 of FIG. 14, the factor to be improved is notified to the target person B. Specifically, the second notification unit 28 notifies the target person B about one or more of the factors from among the factors that is determined to have contributed to improving the target person's blood glucose level, as the factor to be improved, in order to stimulate improving the target person B's lifestyle in relation to the specified factor. For example, the second notification unit 28 notifies the target person B about the above-specified four factors as the factors to be improved.

As described above, according to the second lifestyle improvement support method, the factors to be improved can be proposed to the target person. According to this method, the factors to be improved can be proposed to the target person based on whether the target person's blood glucose level data is improved (i.e., based on the target person's specific data). Therefore, the second lifestyle improvement support method can appropriately support the improvement of each target person's lifestyle.

In addition to the above, a lifestyle modification support program that executes the system disclosed in this specification is provided. More specifically, the lifestyle improvement support program is provided in the controlling device of the lifestyle improvement support system, wherein the program executes: extracting the plurality of factors that the target person should improve and that include the factors related to the target person's physical activity and the factors related to their dietary activity, based on at least the target person's physical activity data and dietary activity data; selecting one or more factors from among the plurality of extracted factors; notifying the target person about the selected one or more factors in order to stimulate the target person to improve their lifestyle in relation to each factor; determining whether each of the factors improved by the target person contributed to improving their blood glucose level based on at least the blood glucose level data acquired from the target person before their lifestyle improvement and the blood glucose level data acquired from the target person after stimulating the target person to improve their lifestyle; and notifying the target person about one or more factors from among the factors determined to contribute to improving the target person's blood glucose level in order to stimulate the target person to improve their lifestyle in relation to each factor. The lifestyle improvement support system is configured to notify the target person about the factor to be improved by the target person based on whether the target person's blood glucose level data is improved (in other words, based on the target person's specific data), and the like. Therefore, the second lifestyle improvement support method can appropriately support the improvement of each target person's lifestyle.

### Modified Embodiment

Although some embodiments of the present disclosure are described above, each of the above embodiments is merely one example thereof. The embodiments of the present disclosure can be realized by various other embodiments. The present disclosure can also be realized in accordance with the teachings disclosed in the present specification and common general knowledge in the art. The technology described in the present claims includes various modifications and changes of the above-described exemplary embodiments. For example, a part of the above-described embodiments can be replaced with other modifications, and other modifications may be added to the above-described embodiments. When a technical feature is not described in the above-described embodiments as essential, the technical feature may be omitted from the above-described embodiments, as appropriate.

For example, in the lifestyle improvement support system described above, the acquisition device 12 is configured to be able to acquire a target person's health data, but the lifestyle improvement support system of the present disclosure is not limited thereto. In the lifestyle improvement support system of the present disclosure, the acquisition device 12 may not be configured to be able to acquire the target person's health data. In other words, even when the target person's health data cannot be considered, it is possible to extract a factor related to the target person's physical activity or a factor related to the target person's dietary activity to be improved by the target person.

For example, in the lifestyle improvement support system described above, the acquisition device 12 is configured to be able to acquire the target person's self-efficacy, but the lifestyle improvement support system of the present disclosure is not limited thereto. In the lifestyle improvement support system of the present disclosure, the acquisition device 12 may not be configured to be able to acquire the target person's self-efficacy. In other words, even when the self-efficacy cannot be considered, it is possible to select instead one or more factors from among the above-specified extracted factors.

For example, in the lifestyle improvement support system described above, the acquisition device 12 and the controlling device 21 are provided in different terminals, but the lifestyle improvement support system of the present disclosure is not limited thereto. The acquisition device 12 and the controlling device 21 may be provided in the same terminal as long as the technical advantages in the present disclosure can be realized.

For example, in the lifestyle improvement support system described above, the determination unit 27 determines whether the target person's improved factor contributed to improving the target person's blood glucose level based on the blood glucose level data of the previous day, but the lifestyle improvement support system of the present disclosure is not limited thereto. The lifestyle improvement support system described above can also perform the determination using the blood glucose level data of the first baseline survey instead of the blood glucose level data of the previous day. That is, for example, in the lifestyle improvement support system, the blood glucose level data on the second day and the blood glucose level data on the third day are compared, but the blood glucose level data related to the first baseline survey and the blood glucose level data on the third day can also be compared. Similarly, the blood glucose level data on the other days can also be compared to the blood glucose level data related to the first baseline survey for the determination.

For example, in the above description, an example of a flow whereby the extraction unit 24 extracts the factors in the lifestyle improvement support system is described, but the lifestyle improvement support system is not limited thereto. The flow may be modified as appropriate as long as the technical advantages in the present disclosure are realized.

For example, in the above description, two kinds of the selection criterion for selecting factors in the selection unit 25 in the lifestyle improvement support system are described, but the lifestyle improvement support system is not limited thereto. The selection criterion may be modified as appropriate as long as the technical advantages in the present disclosure are realized. Alternatively, the selection unit 25 may select the factors randomly.

For example, in the lifestyle improvement support method described above, the blood glucose level data related to the second baseline survey is acquired and the blood glucose level data is checked, but the lifestyle improvement support method is not limited thereto. In the lifestyle improvement support method of the present disclosure, the blood glucose level data related to the second baseline survey might not be acquired. In other words, the factor to be improved can be notified to the target person only by the blood glucose level data related to the first baseline survey and the blood glucose level data related to improving the target person's lifestyle.

For example, in the lifestyle improvement support method described above, the acquisition of the blood glucose level data related to the second baseline survey is performed after completing all the improvements of the target person's lifestyle, but the lifestyle improvement support method is not limited thereto. In the lifestyle improvement support method of the present disclosure, the blood glucose level data related to the second baseline survey may be acquired by performing the second baseline survey after each of the improvements of the target person's lifestyle (or a predetermined improvement of a target person's lifestyle). The lifestyle improvement support system described above can also determine whether each of the factors contributed to improving the target person's blood glucose level, including the blood glucose level data (including the data related to the second baseline survey).

For example, in the first lifestyle improvement support method described above, the improvement of the target person's lifestyle based on a provided factor is performed again when it cannot be confirmed the target person's blood glucose level improved when the target person improved their lifestyle in relation to the same factor, but the lifestyle improvement support method is not limited thereto. For example, from the viewpoint of more quickly notifying the target person about the factor to be improved, even when there is an improvement in the target person's lifestyle but improvement in the blood glucose level is not achieved, the same improvement in the lifestyle based on the same factor may not be performed again. In such case, the lifestyle improvement support method can be implemented in the order of steps S1 to S5, S7, S6, and S9 in FIG. 4.

For example, in the above-described lifestyle improvement support method, only the factor to be improved is notified to the target person on the last day, but the lifestyle improvement support method is not limited thereto. The lifestyle improvement support method of the present disclosure may be configured to transmit the results of the first baseline survey and the results of the lifestyle improvement's contribution to improving the target person's blood glucose level related to the relevant factor, as a report each time.

For example, in the first lifestyle improvement support method and the second lifestyle improvement support method, the baseline surveys (i.e., the first baseline survey and the second baseline survey) and the lifestyle improvement proposals related to the selected factors are performed over several days, respectively, but the lifestyle improvement support method is not limited thereto. For example, in the lifestyle improvement support method of the present disclosure, the baseline survey may be conducted for about one week, and a target person's lifestyle improvement proposal related to a selected factor may be made over several weeks.

Although the specific examples of the present technology are described in detail, they are merely examples and do not limit the scope of the claims. The technology described in the claims includes various modifications and changes of the specific examples described above.

### REFERENCE SIGNS LIST

1 Lifestyle Improvement Support System
10 Target Person's Terminal
20 Management Server
21 Controlling Device
30 Continuous Glucose Meter (continuous glucose monitor)
40 Sensor Device

1. A lifestyle improvement support system that supports improving a target person's blood glucose level by improving the target person's lifestyle, comprising one or more processors and one or more memories, the processor programmed to:
receive target person's blood glucose level data and acquired data including at least target person's physical activity data and dietary activity data, the target person's blood glucose level data and the acquired data being transmitted by a terminal; and
extract a plurality of factors as target factors from among one or more candidate factors including one or more factors related to physical activity and one or more factors related to dietary activity, based on the acquired data, the target factors being for improving target person's lifestyle;
select one or more factors from among the target factors;
perform a first notification process of notifying the target person about selected one or more factors to stimulate the target person to improve their lifestyle in relation to each of the selected one or more factors;
determine whether the selected one or more factors contributed to improving the target person's blood glucose level based on at least first blood glucose level data acquired from the target person before their lifestyle improvement and second blood glucose level data acquired from the target person after the target person was stimulated to improve their lifestyle based on the selected one or more factors notified by the first notification process; and
perform a second notification process of notifying the target person about one or more factors from among the factors determined to contribute to improving the target person's blood glucose level in order to stimulate the target person to improve their lifestyle in relation to each of the factors.

2. The lifestyle improvement support system according to claim 1, wherein the processor is programmed to:
when the acquired data includes health data,
extract the target factors based on at least the physical activity data, dietary activity data and health data included in the acquired data.

3. The lifestyle improvement support system according to claim 1, wherein the processor is programmed to:
receive information of target person's self-efficacy input by the target person, and
perform a third notification process of notifying the target person about the target factors to input the information of the target person's self-efficacy to improve the target person's lifestyle in relation to one or more of the notified target factors, and
select the selected one or more factors from among the notified target factors based on the information of the target person's self-efficacy.

4. (canceled)

5. The lifestyle improvement support system according to claim 1, wherein,
the processor is programmed to:
acquire the second blood glucose level data during executing the target person's physical activity and dietary activity for improving their lifestyle after notifying the selected one or more factors by the first notification; and
determine the selected one or more factors contributed to improving the target person's blood glucose level based on acquired second blood glucose level data.

6. The lifestyle improvement support system according to claim 5, wherein,
the processor is programmed to acquire the second blood glucose level data during executing the target person's physical activity and dietary activity for improving their lifestyle while varying at least one of the type and number of the selected one or more factors for each given period.

7. The lifestyle improvement support system according to claim 1, wherein
the first blood glucose level data is received during a baseline survey period in which the target person does not execute the physical activity and the dietary activity for the lifestyle improvement.

8. The lifestyle improvement support system according to claim 1, wherein
the dietary activity data includes at least one of the types of data relating to calorie of the meal, nutrition included in the meal and intake time of the meal.

9. The lifestyle improvement support system according to claim 1, wherein
the physical activity data includes performance time data of the physical activity of the target person.

10. A lifestyle improvement support method that supports improvement of a blood glucose level by improving a lifestyle of a target person, comprising one or more processors and one or more memories, comprising:
receiving target person's blood glucose level data and acquired data including at least target person's physical activity data and dietary activity data, the target person's blood glucose level data and the acquired data being transmitted by a terminal; and
extracting a plurality of factors as target factors from among one or more candidate factors including one or more factors related to physical activity and one or more factors related to dietary activity, based on the acquired data, the target factors being for improving target person's lifestyle;
selecting one or more factors from among the target factors;
performing a first notification process of notifying the target person about selected one or more factors to stimulate the target person to improve their lifestyle in relation to each of the selected one or more factors;
determining whether the selected one or more factors contributed to improving the target person's blood glucose level based on at least first blood glucose level data acquired from the target person before their lifestyle improvement and second blood glucose level data acquired from the target person after the target person was stimulated to improve their lifestyle based on the selected one or more factors notified by the first notification process; and
performing a second notification process of notifying the target person about one or more factors from among the factors determined to contribute to improving the target person's blood glucose level in order to stimulate the target person to improve their lifestyle in relation to each of the factors.

11. A lifestyle improvement support system that supports improving a target person's blood glucose level by improving the target person's lifestyle, comprising, comprising:
a terminal device; and
a server, wherein
the terminal device acquires target person's blood glucose level data and acquired data including at least target person's physical activity data and dietary activity data, and
the server has one or more processors and one or more memories, the processor programmed to:
receive target person's blood glucose level data and acquired data including at least target person's physical activity data and dietary activity data, the target person's blood glucose level data and the acquired data being transmitted by a terminal; and
extract a plurality of factors as target factors from among one or more candidate factors including one or more factors related to physical activity and one or more factors related to dietary activity, based on the acquired data, the target factors being for improving target person's lifestyle;
select one or more factors from among the target factors;
perform a first notification process of notifying the target person about selected one or more factors to stimulate the target person to improve their lifestyle in relation to each of the selected one or more factors;
determine whether the selected one or more factors contributed to improving the target person's blood glucose level based on at least first blood glucose level data acquired from the target person before their lifestyle improvement and second blood glucose level data acquired from the target person after the target person was stimulated to improve their lifestyle based on the selected one or more factors notified by the first notification process; and
perform a second notification process of notifying the target person about one or more factors from among the factors determined to contribute to improving the target person's blood glucose level in order to stimulate the target person to improve their lifestyle in relation to each of the factors.
